(19) United States
(12) Patent Application Publication
(10) Pub. No.: US 2013/0158364 A1
(43) Pub. Date: Jun. 20, 2013

(54) Device Embodied to Measure Vital Parameters of a Patient

(71) Applicant: Robert Bosch GmbH, Stuttgart (DE)

(72) Inventors: Henning Hayn, Stuttgart (DE); Ulrich Ladstaetter, Weinstadt (DE)

(73) Assignee: Robert Bosch GmbH, Stuttgart (DE)

(21) Appl. No.: 13/711,697

(22) Filed: Dec. 12, 2012

(30) Foreign Application Priority Data
Dec. 16, 2011 (DE) 10 2011 088 817.9

(51) Int. Cl. A61B 5/0205; A61B 5/11; A61B 5/01; A61B 5/107; A61B 5/0402; A61B 5/103; A61B 5/145; A61B 5/087; A61B 5/1455; A61B 5/08; A61B 7/02; A61B 5/00; A61B 5/021

(52) U.S. Cl. CPC A61B 5/0205; A61B 5/00; A61B 5/1116; A61B 5/1121; A61B 5/1124; A61B 5/01; A61B 5/1072; A61B 5/0402; A61B 5/021; A61B 5/14532; A61B 5/087; A61B 5/1455; A61B 5/082; A61B 7/02; A61B 5/0059; A61B 5/4872; A61B 5/4875; A61B 5/0004; A61B 5/103
USPC 600/301

(57) ABSTRACT

A measuring device includes a plurality of measuring modules and a control module. The plurality of measuring modules is configured to measure vital parameters of a patient, with at least two measuring modules of the plurality of measuring modules being configured to measure different vital parameters. The control module is configured to evaluate the measured vital parameters and/or to carry out calculations based on the measured vital parameters.

This application claims priority under 35 U.S.C. §119 to patent application no. DE 10 2011 088 817.9, filed on Dec. 16, 2011 in Germany, the disclosure of which is incorporated herein by reference in its entirety.

### BACKGROUND

The disclosure relates to a device which is embodied to measure vital parameters of a patient.

Known telemedical systems often consist of three components: the patient terminal; the interface for the medical specialist staff, for displaying data and for receiving inputs; and a server by means of which the patient data measured by the patient terminal is captured, processed and provided to the medical specialist staff via the interface. The patient terminals are often various items of peripheral equipment, which are connected to the telemedical system for capturing vital parameters. Vital parameters are well known and constitute indices which reflect the basic functions of the human body. In medicine, they are measured to monitor vital functions. Patient terminals can for example be blood pressure measuring equipment, blood sugar measuring equipment, spirometers, pulse oximeters, body scales, etc. Vital parameters are the data or values measured by the patient terminals. The vital parameters are read out from the patient terminals, transmitted to the server and subsequently made available to the specialist staff via the interface.

However, the problem of conventional telemedical systems is that the capture of vital parameters and the transmission thereof to the specialist staff is cumbersome for the patient and requires much time. There often is the need for painstaking individual configuration of the patient terminals, during which the patient terminals are personalized to the patient (e.g. by specifying the age, the sex, the body height, the weight, etc.). There can often be operating and/or input errors in a multiplicity of patent terminals. As a result of the multiplicity of necessary individual configurations and as a result of the multiplicity of patient terminals which can be operated and/or configured incorrectly, measurement errors which remain undetected often occur. Hence there still is a need for efficient, time-saving and flexible systems and devices, which enable error-free, efficient, flexible and time-optimized establishment and provision of patient data relating to the vital parameters.

### SUMMARY

The object of the present disclosure is to provide an improved device or an improved system, which is configured to establish, measure and/or calculate data relating to vital parameters, and to subsequently provide it.

The concept of the present disclosure lies in providing multifunctional measuring equipment or a multifunctional measuring device, which unifies a multiplicity of items of measuring equipment, measuring modules and/or sensors in one and moreover allows efficient, flexible, comprehensive and fast evaluation of the currently measured, evaluated data, with the data applying to a specific patient and relating to vital parameters.

The present disclosure provides a device, which enables error-free, fast and efficient measurements. Furthermore, the device also enables an error-free and effective evaluation of the measured data, which moreover relates to correct and current information in respect of a patient. Moreover, the device can be designed and produced in a cost-effective manner when it enables and/or supports a modular design of the various measuring processes.

### BRIEF DESCRIPTION OF THE DRAWINGS

In the following figures, the disclosure will schematically be described in more detail on the basis of exemplary embodiments. Here, equivalent elements or elements having the same effect can be provided with the same reference sign for reasons of clarity.

FIG. 1 shows a device for measuring vital parameters according to one exemplary embodiment of the present disclosure;

FIG. 2 shows a device for measuring vital parameters according to one exemplary embodiment of the present disclosure;

FIG. 3 shows a device for measuring vital parameters according to one exemplary embodiment of the present disclosure;

FIG. 4 shows a device for measuring vital parameters according to one exemplary embodiment of the present disclosure;

FIG. 5 shows a device for measuring vital parameters according to one exemplary embodiment of the present disclosure;

FIG. 6 shows a device for measuring vital parameters according to one exemplary embodiment of the present disclosure;

FIG. 7 shows a device for measuring vital parameters according to one exemplary embodiment of the present disclosure;

FIG. 8 shows a device for measuring vital parameters according to one exemplary embodiment of the present disclosure;

FIG. 9 shows a finger-slot measuring module according to one exemplary embodiment of the present disclosure; and

FIG. 10 shows a respiratory gas analysis module according to one exemplary embodiment of the present disclosure.

### DETAILED DESCRIPTION

The figures relate to various aspects of the configuration of the device. It is therefore possible to combine together the exemplary embodiments of the device which are shown in the figures and described in the application. In the figures, this is clarified, inter alia, by the use of the same or similar reference signs.

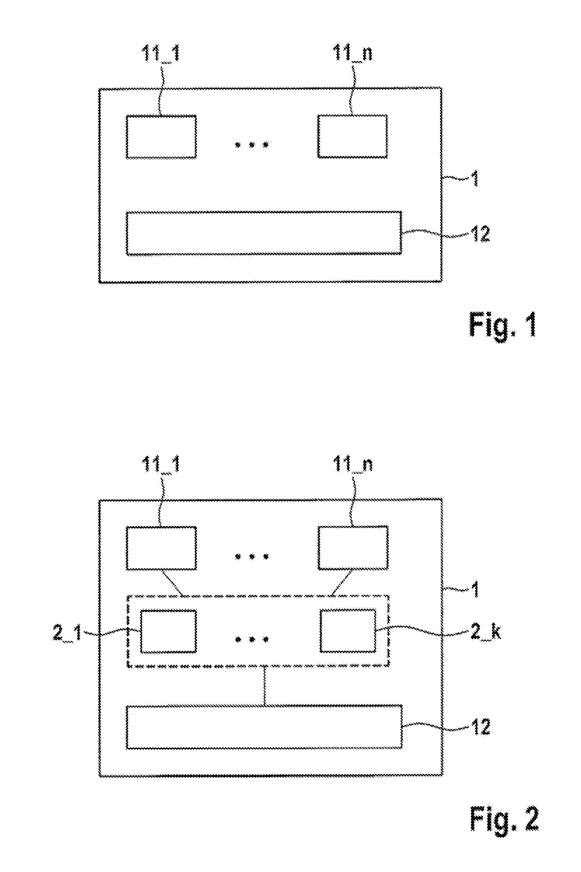

FIG. 1 shows a device 1 for measuring vital parameters of a patient according to one exemplary embodiment of the present disclosure. Here the device 1 has a number of measuring modules 11_1 to 11_{—}n, which are designed to measure vital parameters. According to the present exemplary embodiment, at least two measuring modules 11_1 to 11_{—}n respectively measure different vital parameters. According to the present exemplary embodiment, the number of measuring modules 11_1 to 11_{—}n is at least two measuring modules, i.e. n is a natural number greater than or equal to two. By way of example, vital parameters can be at least one of the following values measured at the patient: vital parameters measured by an electrocardiogram (EKG); temperature; weight; body fat percentage; body water percentage; (body) height; balance; body center of gravity; sense of poise; coordination; blood pressure; concentration of constituents of the respiration; blood sugar. Here, according to the present disclosure, it is also possible to measure further vital parameters not listed above. Rather, the vital parameters listed above constitute an exemplary and not an exhaustive list. By way of example, measuring modules 11_1 to 11_{—}n can have one of the following measuring modules or sensors: a weight measuring module; a balance measuring module; a body center of gravity measuring module; a coordination measuring module; a temperature measuring module, wherein the temperature measuring module can measure the temperature of the lower extremities, the upper extremities or on the forehead; a body height measuring module; an EKG measuring module; a blood pressure measuring module; a blood sugar measuring module; a spirometry measuring module; a measuring module for photometric blood analysis; a measuring module for respiratory gas analysis; a stethoscope; an impedance measuring module, wherein the impedance measuring module can measure the impedance of the lower extremities or upper extremities; an optical examination module (e.g. a camera), which can examine the skin and/or body parts using image-processing units. In this respect, it should be noted that the measuring modules 11_1 to 11_{—}n listed above are only exemplary and have not been listed in an exhaustive fashion. Thus, the device 1 according to the present disclosure can also have further measuring modules 11_1 to 11_{—}n, which are not listed above.

Furthermore, the device 1 according to the present exemplary embodiment has a control module 12, which is designed to evaluate the measured vital parameters and/or to carry out calculations on the basis of the measured vital parameters. Hence the evaluation of the measured information or vital parameters is respectively carried out in the device 1 itself without the need for respectively transmitting the information or vital parameters to a further device specifically designed to evaluate this data.

The measuring modules 11_1 to 11_{—}n themselves can have various designs, with the design variety thereof being known to a person skilled in the art. By way of example, the weight measuring module can be a set of scales. By way of example, if use is made of medical scales, it is also possible to capture and measure the body height of a patient in addition to the weight of the patient. Overall, the device 1 or the control module 12 is able to capture or calculate further information or data in respect of the patent from the measured vital parameters. By way of example, the vital parameters weight and body height can be used to calculate the body mass index (BMI). If use is made of body-fat scales or body-water scales, it is also possible (e.g. using a bioelectric impedance analysis (BIA)) to establish or measure the percentage of the body water and of the body fat as further vital parameters, in addition to the weight. Furthermore, scales-like systems can be used as the weight measuring module. Such scales-like system can respectively have at least one integrated force sensor or at least one integrated force measuring module, in order to measure the body weight and the weight distribution as vital parameters. By way of example, it is possible to draw conclusions or determine or calculate information in respect of the body balance of the patient from the measured body weight and the measured weight distribution. The device 1 can also have a plurality of different measuring modules 11_1 to 11_{—}n of the same type. By way of example, the device 1 can have a plurality of different sets of scales.

As presented above, a measuring module 11_1 to 11_{—}n can measure more than one vital parameter. An EKG measuring module can, by means of further integrated sensors, for example additionally also measure vital parameters such as the oxygen saturation (SpO₂), the carbon monoxide content (SpCO), the level of methemoglobin (SpMet), the expiratory carbon dioxide content (MetCO₂) and/or the blood pressure (non-invasive/invasive (NIBP/IBP)).

The temperature measuring module or the thermometer can respectively likewise have various designs, for example in respect of how the measurement is taken. It is possible to carry out oral, axillary, rectal, vaginal, inguinal measurements using an analog or digital peak-value thermometer or measuring module. An ear thermometer makes it possible to carry out the measurement by measuring the infrared emissions from the eardrum. Furthermore, the temperature can also be measured by means of a contact measurement on the forehead or by means of a contactless surface thermometer (e.g. on the forehead).

By way of example, the design of the blood pressure measuring modules can depend on whether an invasive or non-invasive measuring method is used. In the case of invasive measurement of the blood pressure, there is arterial access to the bloodstream, with the access being secured by appropriately applying a catheter. The catheter is then connected to the blood pressure measuring module for measuring the blood pressure. In the case of a non-invasive measurement of the blood pressure, the measurement can be brought about by auscultation (by means of a blood pressure cuff that can be pumped manually and stethoscope), palpation (by means of a blood pressure cuff that can be pumped manually and by means of feeling the pulse), oscillation (electronically) or via the change in the pulse transit times.

Measuring modules for the respiratory gas analysis are designed to analyze the human respiratory air. In the process, it is possible to identify marker substances, which allow conclusions to be drawn in respect of the clinical state of a patient. By way of example, it is possible to determine concentrations of CO, CO₂, H₂, and C₂H₆O in the respiratory air. Each measured substance concentration can be considered to be a vital parameter. The measured concentrations can then be used to convert the respective respiratory gas concentrations into corresponding blood concentrations (as further information obtained from the vital parameters). According to the present exemplary embodiment, these calculations are carried out by the device 1 or by the control module 12. Measuring the concentrations themselves is well established and known to a person skilled in the art.

The device 1 can have such a modular design that the device permits the following: the removal of the measuring modules 11_1 to 11_{—}n; the replacement of the measuring modules 11_1 to 11_{—}n; and/or the addition of new measuring modules 11_1 to 11_{—}n. As a result of this modular design, the device 1 can have a patient-individual design since not every patient requires all measuring modules 11_1 to 11_{—}n. As a result, it is also possible to save costs when providing the measuring modules 11_1 to 11_{—}n to a patient and/or when producing the device 1. If there is a change in the health situation (e.g. as a result of an occurring comorbidity or necessary temporary postoperative monitoring), additional measuring modules 11_1 to 11_{—}n can be attached (also on a temporary basis) in a simple fashion. The measuring modules 11_1 to 11_{—}n can likewise also be removed or replaced. The device 1 can be (modularly) connected or attached to at least one of the measuring modules 11_1 to 11_{—}n, for example via a (suitable) quick-release fastener. By way of example, at least one of the quick-release fasteners can be a plug-in connection, a sliding connection, a screw connection or a clamping connection, wherein use can also be made of other quick-release fasteners that are suitable for this. A person skilled in the art generally knows of quick-release fasteners.

The device 1 can have at least one interface for connecting the measuring modules 11_1 to 11_{—}n to the control module 12. This further design of the device 1 is shown in FIG. 2. The interfaces 2_1 to 2_{—}k can have different designs. Thus, for example, each measuring module 11_1 to 11_{—}n can be connected to the control module over a specific interface 2_1 to 2_{—}k. Furthermore, there can be interfaces 2_1 to 2_{—}k which connect a plurality of measuring modules 11_1 to 11_{—}n (i.e. more than one measuring module 11_1 to 11_{—}n) to the control module 12. Moreover, there can also be a single interface 2_1 to 2_{—}k for connecting the measuring modules 11_1 to 11_{—}n to the measuring module 11_1 to 11_{—}n. That is to say k is a natural number which is greater than or equal to one. The interfaces 2_1 to 2_{—}k serve to transfer data between the control module 12 and the measuring modules 11_1 to 11_{—}n. Here, the interfaces 2_1 to 2_{—}k are designed to transfer the vital parameters measured by the measuring modules 11_1 to 11_{—}n to the control module 12. According to one exemplary embodiment, the individual measuring modules 11_1 to 11_{—}n can communicate via a data bus (e.g. CAN bus). Here the control module 12 has interfaces 2_1 to 2_{—}k to the bus system for the measuring modules 11_1 to 11_{—}n. According to a further exemplary embodiment, the communication from the measuring modules 11_1 to 11_{—}n to the control module 12 can take place by means of e.g. interfaces 2_1 to 2_{—}k, of which at least one of the interfaces 2_1 to 2_{—}k can be one of the following interfaces: Bluetooth, ZigBee, infrared interface, cabled connection (e.g. USB, RS 232, other type of cabled connection). Here, it is also possible to use other interfaces 2_1 to 2_{—}k that are suitable and known to a person skilled in the art.

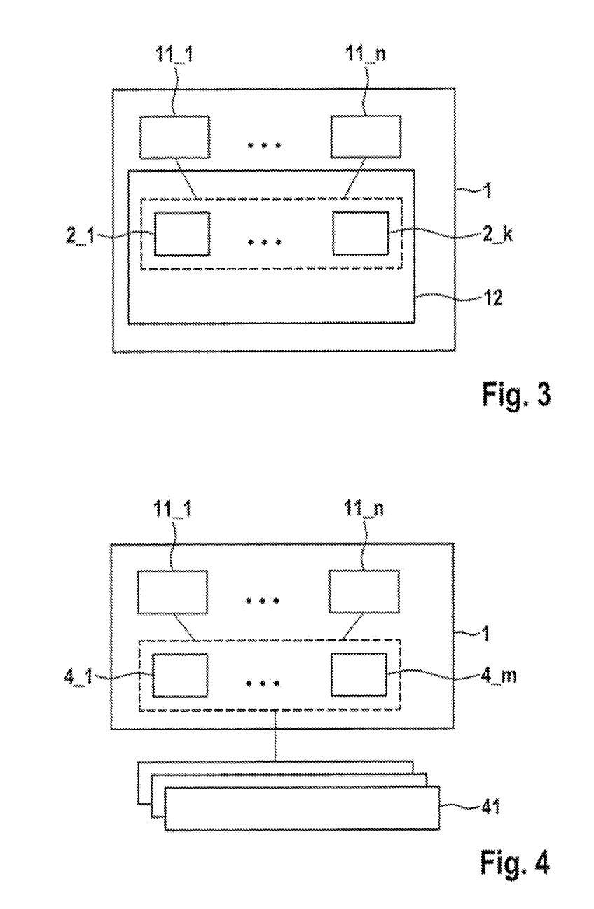

Furthermore, a mixed form of the two exemplary embodiments is also possible, i.e. some of the measuring modules 11_1 to 11_{—}n can be connected to the control module 12 via a data bus and some of the measuring modules 11_1 to 11_{—}n can be connected thereto via a different type of interface 2_1 to 2_{—}k (as listed above in an exemplary fashion). Moreover, the interfaces can be arranged in and/or on the control module 12, as shown in FIG. 3.

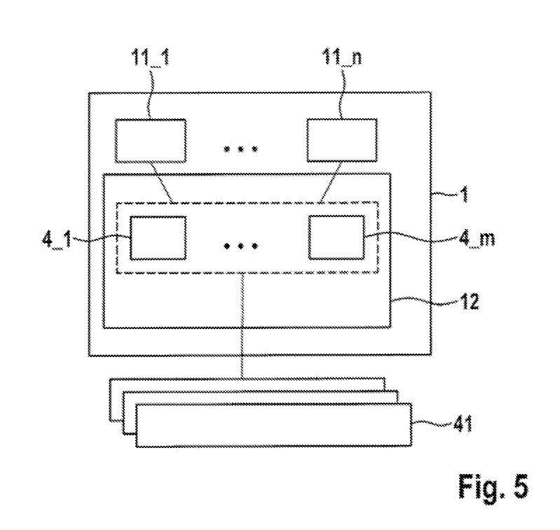

The device 1 can be designed to communicate with at least one further (medical) device 41. This is illustrated in FIG. 4 in an exemplary fashion, wherein the device 1 moreover can also be designed as illustrated in FIGS. 1 to 3 in an exemplary fashion. In order to communicate with further devices 41, the device 1 can have further interfaces 4_1 to 4_{—}m, which enable the communication between the device 1 and the at least one further device 41. These interfaces 4_1 to 4_{—}m can have a similar design to the above-described interfaces 2_1 to 2_{—}k. Thus, an interface 4_1 to 4_{—}m can connect one, some or all of the further (medical) devices 41 to the device 1 for the transmission of data. At least one of the interfaces 4_1 to 4_{—}m can be one of the following interfaces: Bluetooth, infrared interface, cabled connection (e.g. USB, RS 232, other type of cabled connection). It is also possible to use other interfaces which are suitable for this. A person skilled in the art is generally aware of such interfaces. The further interfaces 4_1 to 4_{—}m can be arranged in and/or on the control module 12, as shown in FIG. 5 in an exemplary fashion, wherein the device 1 in FIG. 5 can also be designed as illustrated in an exemplary fashion in FIGS. 1 to 3.

The control module 12 can be designed to calculate characteristics and/or further information in respect of the patient, in respect of whom the measurements are carried out by the device 1, on the basis of the measured vital parameters.

Characteristics and the fundamentals of calculating the latter are well known. In general, a characteristic is an index serving to quantify the measured vital parameters. The characteristic is based on a prescription/definition for the quantitatively reproducible measurement of the measured vital parameters. The characteristics can be defined in the device 1 or in the control module 12. After measuring the vital parameters, the characteristics are calculated by the device 1 or the control module 12. The calculated characteristics can display further information in respect of the state of the patient and/or can be used to calculate further information in respect of the state of the patient. In the following text, a few characteristics and their use are listed in an exemplary (but not exhaustive) fashion. An exemplary characteristic can specify the BMI, which is calculated according to the following formula: BMI=m/l², where m specifies the body mass and l specifies the body height. At least one of the two values m and l can be measured as vital parameter by the measuring modules 1_1 to 1_{—}n of the measuring system, i.e. the device 1. The characteristic specifying the BMI can be calculated by the device 1 or the control module 12. A further exemplary characteristic can specify the Broca index, which can be calculated by means of the formula mₙₒᵣₘ=(1-100) kg, with the Broca index defining the normal weight mₙₒᵣₘ of a person via the height l and with it being possible for the height l, as a vital parameter, to have been measured by means of the measuring modules 1_1 to 1_{—}n of the device 1. According to the present disclosure, any number of further characteristics can be defined and/or calculated. Here, it is possible, for example, for the measured vital parameters to be put in relation to one another or in relation to the age and/or to other patient-specific factors, parameters, values and specifications. The characteristics can be compared to predetermined (limit) values, which can for example be stored (e.g. in tables) in the device 1 or in the control module 12, and can as a result of this display the state of the patient. Such further characteristics can display values which are generally used under the term fitness score. It is also possible to form characteristics from the relation of various gas components in the respiratory air. Furthermore, it is possible to define and/or calculate characteristics which reproduce a risk assessment of the patient. A characteristic formed from e.g. weight, body water percentage and blood pressure can provide an indication of an upcoming exacerbation in the case of patients with chronic obstructive pulmonary disease (COPD). A person skilled in the art is aware of the breadth of variation when defining and calculating characteristics. The present disclosure can therefore be used in respect of any suitable characteristic.

As already illustrated above in an exemplary fashion, it is possible, for example, to calculate the BMI as characteristic from the vital parameters such as weight and body height, or it is possible calculate corresponding blood concentrations as characteristics from the vital parameters such as the measured respiratory gas concentrations, which characteristics can be connected to further specifications of the state of the patient (e.g. there may be further risks in the case of a specific BMI). The further specifications for example constitute such further information, with it also being possible to determine other information from the measured vital parameters. A person skilled in the art is well aware of how to calculate further information on the basis of the vital parameters.

As mentioned previously, the device 1 can be designed to communicate with at least one further device 41. In the process, the device 1 can transmit the vital parameters, characteristics and/or other information calculated on the basis of the measured vital parameters to the further devices 41. The further devices 41 can be designed to receive the vital parameters, characteristics and/or other information calculated on the basis of the measured vital parameters from the device 1 and moreover to store them, process them, evaluate them, use them for other calculations, display them and/or transfer them. The device 1 can furthermore be designed to receive data or information from the at least one further device 41, wherein this data or information can be data or information relating to e.g. at least one patient, for example relating to the patient in respect of whom the device 1 carries out the measurements. This data or information can for example have predetermined limits in respect of the calculated characteristics and/or in respect of the measured vital parameters, which limits can also be e.g. patient-specific (e.g. related to the patient to be measured). As a result of the comparison of the characteristics and/or the vital parameters with the limits predetermined on a patient-individual basis (by means of the device 1 or by means of the control module 12), the device 1 can immediately, and without first having to transfer data to the at least one further device 41, give the patient feedback in respect of the measured values, e.g. by a suitable indication (e.g. visually, acoustically, speech).

As a result of measuring a plurality of vital parameters, it is also possible to carry out plausibility tests in respect of individual vital parameters and/or characteristics by means of the device 1 or by means of the control module 12. By way of example, the concurrent increase of body weight and body water percentage can indicate water retention (e.g. in the case of COPD (chronic obstructive pulmonary disease) patients). The results of such plausibility tests, which calculate relationships between the vital parameters, the characteristics and/or the further information calculated from the vital parameters can be indicated by the device 1, e.g. by means of a suitable indication (e.g. visually, acoustically, speech), and/or transferred to at least one further device 41.

The device 1 or the control module 12 can be designed to identify the patient, on whom the measurements should be undertaken, on the basis of patient-specific characteristics which were calculated on the basis of the measured vital parameters in respect of the patient. By way of example, this is expedient when the device 1 is used by a plurality of patients/users. The identification of the user e.g. exclusively via the weight as one vital parameter is not always unique. As a result of the combination with other vital parameters, i.e. on the basis of calculated characteristics, the device 1 or the control module 12 can uniquely identify the patient (even without an additional biometric sensor). To this end, the device 1 can have an entry module, by means of which the respective characteristic identifying the patient can be entered. After identifying the patient, the device 1 or the control module 12 can be designed to link the measured vital parameters, the characteristics calculated from the measured vital parameters and/or further information to the identified patient, for whom the measurements were carried out.

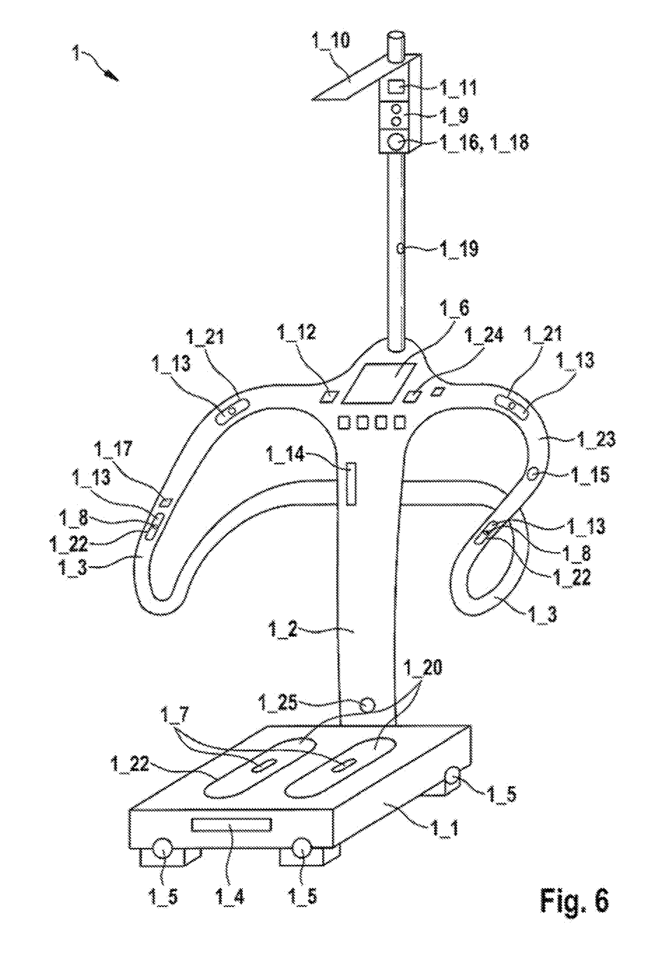

FIG. 6 shows the device 1 for measuring vital parameters according to a further exemplary embodiment of the present disclosure, in which a more specific design of the device 1 with the modules 11_1 to 11_{—}n has been shown in an exemplary fashion. It is understood that, according to the present disclosure, other more specific designs of the device 1 with the modules 11_1 to 11_{—}n are also possible. The device 1 can also only have some of the modules from FIG. 6 and/or can have other modules as well.

According to the present exemplary embodiment, the device 1, which is embodied as an integrated, modular measuring system for vital parameters, has a scales-like base module 1_1, which stands on the ground, and the following modules: a central column 1_2; handles 1_3; for the base module 1_1, respectively a force sensor or a force measuring module 1_4 which can be designed to measure the weight; for the base module 1_1, a plurality of further force sensors or force measuring modules 1_5, which can be designed to measure the balance, body center of gravity, coordination; a human-machine interface 1_6 (e.g. display, keys, loudspeakers); for lower extremities, temperature sensors or temperature measuring modules 1_7 which can be installed in the base module 1_1; for upper extremities, temperature sensors or temperature measuring modules 1_8 which can be installed in the handles 1_3 of the device 1; a temperature sensor or temperature measuring module 1_9 for the forehead (e.g. contactless via infrared); a measuring apparatus for the body height 1_10 (via a mechanical system or by optical means); an additional display or indication module 1_11 (e.g. for installation into the measuring apparatus for the body height); an EKG module 1_12 (e.g. with a connector for 12-channel electrodes); dry electrodes 1_13 (e.g. for installation into the handles 1_3 of the device 1); a blood pressure measuring module 1_14; a blood sugar measuring module 1_15; a spirometry measuring module 1_16; a module for photometric blood analysis 1_17 (e.g. a finger slot for pulse oximetry); a respiratory gas analysis module 1_18; a microphone 1_19 (e.g. electronic stethoscope); for the lower extremities, an impedance sensor or an impedance measuring module 1_20 which can be installed in the base module 1_1; for upper extremities, an impedance sensor or an impedance measuring module 1_21 which can be installed in the handles 1_3 of the device 1; for optical feedback, a light source 1_22 which can be installed in the handles 1_3 or in the base module 1_1 of the device 1; for haptic feedback, actuators 1_23 which can be installed in the handles 1_3 or in the base module 1_1 of the device 1; a biometric sensor or a biometric measuring module 1_24 for identifying and authenticating the user (e.g. a fingerprint sensor); a camera 1_25 for the optical examination of skin or body parts on the basis of image-processing modules or units (e.g. for examining diabetic necrosis on the feet).

The base module 1_1 in FIG. 6 corresponds to the control module 12 explained above. The base module 1_1 in FIG. 6 or the control module 12 respectively is a central control unit of the device 1. The base module 1_1 or the control module 12 can respectively have: a control element (e.g. a microcontroller) for instrument and/or module control and for evaluating the measured vital parameters; an energy supply; interfaces 2_1 to 2_{—}k for the individual modules 1_2 to 1_25 and interfaces 4_1 to 4_{—}m for communication with further (medical) devices (e.g. a telemedical platform/device), wherein the communication can be implemented by means of Ethernet, modem, GSM, UMTS etc.

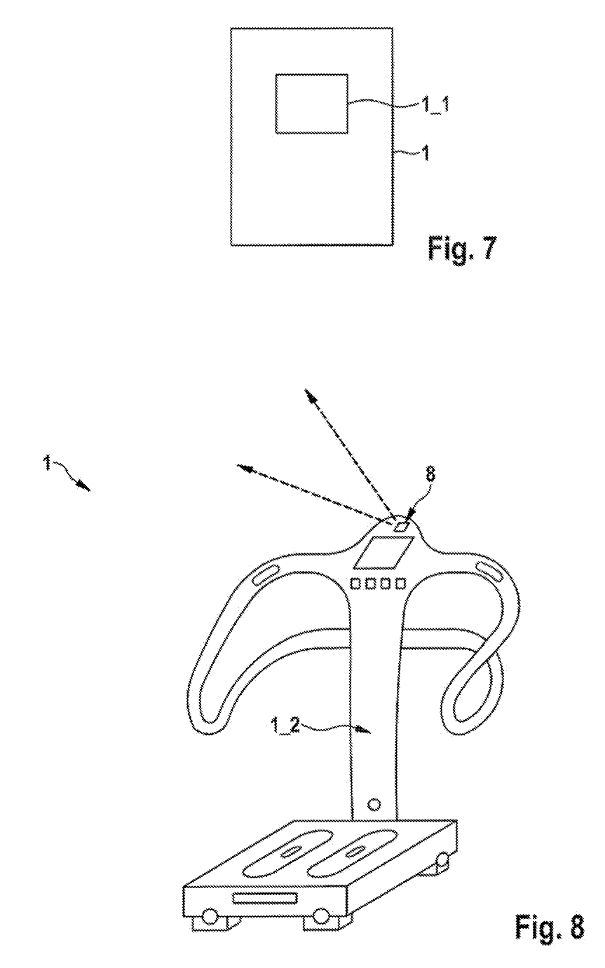

The modules 1_1 to 1_25 can, where necessary, be combined amongst themselves as desired, to the extent that this is possible from a construction point of view. If the body scales function is not required, the base module 1_1 in FIG. 6 can also be placed or arranged elsewhere in the device 1 (e.g. not standing on the ground, as illustrated in FIG. 7 in an exemplary fashion). The base module 1_1 can also be arranged in the device 1 and designed such that it is suitable for physically disabled patients (e.g. for wheelchair users). In the process, it is ensured that the implementation and/or the installation of the base module 1_1 is such that the relevant measurement sites of the respective patient can be accessed.

According to the present exemplary embodiment, the temperature sensors or temperature measuring modules 1_7 for the lower extremities (i.e. feet) can be arranged in the treads in the base module 1_1. Using the surface temperatures of the extremities measured by the temperatures sensors and/or temperature measuring modules 1_7, it is possible to draw conclusions, e.g. in the case of diabetes diseases, in respect of the perfusion of the lower extremities in order then to determine a lack of perfusion such as diabetic necrosis at an early stage. The conclusions can (at least in part) be drawn in the base module 1_1 and/or in a further device (after the transmission of the measured temperatures and/or other relevant information, characteristics), such as e.g. a telemedical platform.

As shown in FIG. 6, the device 1 can, according to the present exemplary embodiment, have a biometric sensor or a biometric measuring module 1_24 for the reliable identification of the patient (e.g. via fingerprints).

According to the present exemplary embodiment, the human-machine interface (HMI) 1_6 can be arranged centrally at the upper end of the central column Said interface has a (e.g. LCD, TFT, OLED, etc.) display and optionally operating keys to execute inputs. The HMI 1_6 can also be arranged differently in the device 1; here it should, in general, be positioned such that it can easily be reached and/or is visible by the patient and/or by the other operator of the device. The HMI 1_6 can also have a touch display or touchscreen.

The patient/user can obtain (precise) instructions/indications by means of the HMI 1_6 in respect of which measurement(s) is/are currently being carried out. Furthermore, the patient/user can obtain (precise) instructions/indications by means of the HMI 1_6 in relation to the behavior in view of and/or during the measurement(s). The instructions/indications can, for example, be provided by acoustic, haptic and/or optical means (e.g. by text, videos and/or images, etc.).

The regions/areas of the sensors/measuring modules to be touched, such as e.g. the tread onto which the foot of the patient should be placed, the contact areas for the upper extremities, can for example be indicated by means of light sources 1_22. By way of example, a light source 1_22 can have OLEDs, LEDs, incandescent lamps and/or glow lamps. Other embodiments of the light sources 1_22 are also possible.

Placement and/or operating errors can be indicated graphically, haptically (e.g. by means of vibration etc.) and/or acoustically (e.g. by means of signal tone, speech output, etc.). By way of example, this can be brought about by means of the HMI 1-6. A correct or wanted placement and/or operation can also be indicated, e.g. by means of the HMI 1_6 and/or the light sources 1_22.

The central column 1_2 can have an extendable design. By extending the central column 1_2, it is possible to expand the device 1 by further sensors/measuring modules, e.g. by sensors/measuring modules for the body height 1_10 and forehead temperature 1_9, as shown in an exemplary fashion in FIG. 6. It should be noted here that the central column 1_2 can be extended to complement the device 1 with other sensors/measuring modules as well. In order to expand the central column 1_2 by further sensors/measuring modules, a carriage can be applied to the extension of the central column 1_2. A bracket can be arranged on the central column 1_2 and it can approach the head surface automatically by means of a drive or manually. A sensor can be used to stop the bracket just above the head or when it touches the head (e.g. via ultrasound, optical means etc.). The body height can be established from the position of the carriage on the central column (path sensors). A display can be integrated into the carriage and it is designed to display the measured body height (e.g. in cm, inches or in another unit). By way of example, the display can be an LCD, TFT, OLED or another type of displays or display devices.

Sensors/measuring modules can also be arranged in the carriage itself. Thus, for example, a holder device for an integrated temperature sensor and/or for an integrated temperature measuring module 1_9 can be placed in the carriage. The temperature sensor or the temperature measuring module 1_9 can detect/measure the surface temperature on the forehead of the patient by means of a thermometer (e.g. pyrometer).

FIG. 8 shows the device 1 for measuring vital parameters as per a further exemplary embodiment of the present disclosure. The design of the device 1 of FIG. 8 can substantially be similar to the device 1 of FIG. 6. Using the device 1 from FIG. 8, it is possible to measure the forehead temperature and the body height measurements from the central column 1_2. The respective sensors/measuring modules 8 are arranged in the central column 1_2. By way of example, the forehead temperature can be measured by means of a pyrometer 8. By way of example, the body height can be measured by means of a laser-distance sensor (or a laser-distance measuring module) and an associated calculation method or by means of an optical system (e.g. camera).

Furthermore, the base module 1_1 with the central column 1_2 can be complemented by a connection option for the blood pressure measurement 1_14. The connector for measuring blood pressure 1_14 can for example be a connector for receiving the measured value from peripheral equipment (e.g. a device which is built outside of the device 1, connected to the device 1 via the connector and designed to measure blood pressure) such as a Bluetooth connector or a connector for a pressure tube for the oscillatory measurement.

In the device 1, the base module 1_1 can be expanded by the central column 1_2, an EKG module 1_12 and a blood pressure measuring module 1_14 and by a module for carrying out a method for pulse transit time measurement. To this end, a slot 1_17 for respectively one finger, for measuring pulse waves, can be embodied on the left-hand and right-hand sides of the handle frame.

The one, or both, slots 1_17 can be designed to undertake or at least aid a photometric analysis of the blood. In the process, the following vital parameters, for example, can be captured/measured: oxygen saturation (SpO₂); the carbon monoxide content in the blood (SpCO); level of methemoglobin (SpMet); and/or further measurement or vital parameters, which can be captured by photometric means.

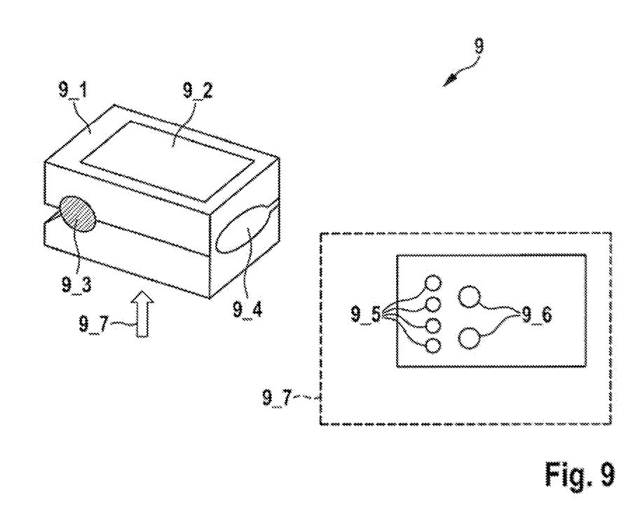

According to a further embodiment, the slot 1_17 can be embodied as a further peripheral device, which can be clipped or attached to a finger and can therefore also be operated independently of the device 1. The slot 1_17 designed as a peripheral device can be connected to the device 1 by means of e.g. Bluetooth, infrared, cabled connections (e.g. by means of USB, RS 232, etc.) or by means of plugging the photometric analysis equipment into a contact point of the device 1. By way of example, the contact point can be arranged in the handle frame of the device 1. In an exemplary fashion, FIG. 9 shows a finger-slot sensor or a finger-slot measuring module 9 as the slot 1_17, which is embodied as a peripheral device. According to the exemplary embodiment of FIG. 9, the finger-slot measuring module 9 has the following components: a body 9_1, a display or a screen 9_2, a multifunction switch or a multifunction joint 9_3, a slot 9_4 for a finger, contact points 9_5 and infrared diodes 9_6, wherein the contact points 9_5 and the infrared diodes 9_6 can be arranged on the rear side 9_7 of the finger-slot measuring module 9.

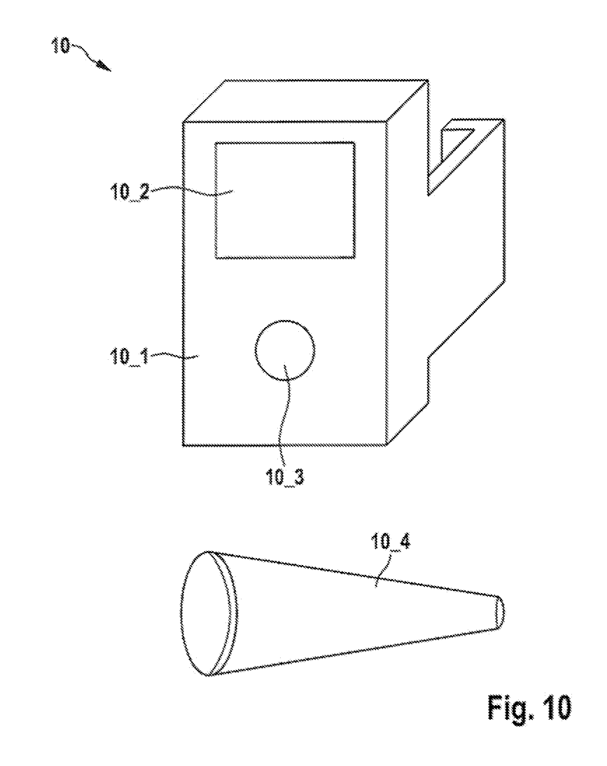

The base module 1_1 with the central column 1_2 and with the extension of the central column 1_2 of the device 1 can be complemented by a height-adjustable respiratory gas analysis module 1_18. The central column 1_2 of the device can be designed such that the respiratory gas analysis module 1_18 can be displaced manually or automatically on the central column 1_2. FIG. 10 shows an exemplary respiratory gas analysis module 10 (provided with the reference sign 1_18 in FIG. 6), as can be used to complement the device 1. According to the exemplary embodiment of FIG. 10, the respiratory gas analysis module 10 can have the following components: a housing 10_1 with a receptacle hole 10_3 for the mouthpiece 10_4, a communication unit for data interchange with the base module 1_1 and with the sensor unit. The respiration analysis carriage can be used to detect various respiratory gases, e.g. CO, CO₂, H₂, C₂H₆O and/or other relevant compounds, the detection of which in this context is known to a person skilled in the art. The respiratory gas analysis module 10 can have a display or a screen 10_2 on which the measured values or vital parameters are indicated with specifications in respect of present respiratory gases (and the concentrations thereof) and/or on which there is an indication whether the blown-in amount of air is sufficient (e.g. by realizing a traffic-light system or in a similar fashion).

Furthermore, sub-regions of the handles 1_3 or of the handle frame and/or parts of the platform of the device 1 can be made of solid metal, and so the whole surface of the parts formed from solid metal can be used as a dry electrode 1_13. The electrodes can likewise be used for a body impedance measurement in order to determine water retention e.g. in the legs or in the lung and in order to determine the cardiac output.

In general, the device 1 can be adapted and optimized for various patient groups. The sensors or the measuring modules can be selected depending on the relevant disorders and/or the examinations to be carried out and can be used with the device 1. Furthermore, the device 1 can be designed for various patient groups, e.g. for patients unable to walk, patients who only have limited ability to walk and/or lying-down patients.

What is claimed is:

1. A measuring device, comprising:
a plurality of measuring modules configured to measure vital parameters of a patient, with at least two measuring modules of the plurality of measuring modules being configured to measure different vital parameters; and
a control module configured to evaluate the measured vital parameters and/or to carry out calculations on the basis of the measured vital parameters.

2. The measuring device according to claim 1, wherein:
the measuring device has a modular configuration,
the plurality of measuring modules are removable from the measuring device,
the plurality of measuring modules are replaceable, and/or
at least one new measuring module is addable to the measuring device.

3. The measuring device according to claim 1, further comprising:
a plurality of interfaces configured to connect the measuring modules to the control module.

4. The measuring device according to claim 1, wherein the control module is further configured (i) to calculate characteristics based on the measured vital parameters, and/or (ii) to calculate further information in respect of the patient based on the measured vital parameters.

5. The measuring device according to claim 4, wherein:
the control module is further configured to compare the calculated characteristics to predetermined limits, and
the measuring device is configured to display a result of the comparison.

6. The measuring device according to claim 4, wherein the measuring device is further configured to identify the patient based on patient-specific characteristics which were calculated based on the measured vital parameters in respect of the patient.

7. The measuring device according to claim 6, wherein the measuring device is further configured to link the measured vital parameters to the identified patient after the patient has been identified.

8. The measuring device according to claim 1, wherein the plurality of measuring modules includes at least one of the following measuring modules:
a weight measuring module,
a balance measuring module,
a body center of gravity measuring module,
a coordination measuring module,
a temperature measuring module,
a body height measuring module,
an EKG measuring module,
a blood pressure measuring module,
a blood sugar measuring module,
a spirometry measuring module,
a measuring module for photometric blood analysis,
a measuring module for respiratory gas analysis,
a stethoscope,
an impedance measuring module, and
an optical examination module,
the temperature measuring module is configurable to measure the temperature of the lower extremities, the upper extremities, or on the forehead,
the impedance measuring module is configurable to measure the impedance of the lower extremities or upper extremities, and
the optical examination module is configured to examine skin and/or body parts using image-processing units.

9. The measuring device according to claim 1, wherein the vital parameters indicate at least one of the following items of information:
vital parameters measured by an EKG,
temperature,
weight,
body fat percentage,
body water percentage,
height,
balance,
body center of gravity,
sense of poise,
coordination,
blood pressure,
concentration of constituents of the respiration, and
blood sugar.

10. The measuring device according to claim 1, wherein:
the measuring device is configured to transmit measured vital parameters, characteristics calculated based on the measured vital parameters, and/or further information in respect of the patient calculated based on the measured vital parameters to a further measuring device and/or to receive data from the further measuring device,
the data comprises data directed at the patient and/or predetermined limits in respect of the calculated characteristics, and
the limits are specific to the patient.
